(19) United States
(12) Patent Application Publication
(10) Pub. No.: US 2020/0063273 A1
(43) Pub. Date: Feb. 27, 2020

(54) System And Process For The Production Of Renewable Fuels And Chemicals

(71) Applicant: Dioxide Materials, Inc., Boca Raton, FL (US)

(72) Inventor: Richard I. Masel, Boca Raton, FL (US)

(21) Appl. No.: 16/400,769

(22) Filed: May 1, 2019

Related U.S. Application Data
(63) Continuation-in-part of application No. 15/684,548, filed on Aug. 23, 2017, now Pat. No. 10,280,378.
Application No. 15/684,548 is a continuation-in-part of application No. 15/158,227, filed on May 18, 2016, now Pat. No. 9,945,040.
Application No. 15/158,227 is a continuation-in-part of application No. 14/704,935, filed on May 5, 2015, now Pat. No. 9,370,773.
Application No. 15/684,548 is a continuation-in-part of application No. 15/400,775, filed on Jan. 6, 2017, now Pat. No. 9,849,450.
Application No. 15/400,775 is a continuation-in-part of application No. 15/090,477, filed on Apr. 4, 2016, now Pat. No. 9,580,824.
(60) Provisional application No. 62/380,917, filed on Aug. 29, 2016.

(51) Int. Cl. C25B 9/18; C10G 2/00; C07C 1/04; C07C 29/152; C07C 41/01; B01J 19/24; B01J 29/40; B01J 29/85; B01J 35/10; C25B 1/04; C25B 9/10; C25B 15/02; C25B 11/04

(52) U.S. Cl. CPC C25B 9/18; C10G 2400/08; C07C 1/041; C07C 29/152; C07C 41/01; B01J 19/245; B01J 29/40; B01J 29/85; B01J 35/1019; C25B 1/04; C25B 9/10; C25B 15/02; C25B 11/0405; C25B 11/0415; C25B 11/0489; C10G 2400/02; C10G 2400/04; C10G 2/35

(57) ABSTRACT

A renewable fuel production system includes a carbon dioxide capture unit for extracting carbon dioxide from atmospheric air, a carbon dioxide electrolyzer for converting carbon dioxide to carbon monoxide, a water electrolyzer for converting water to hydrogen, a synfuels generator for converting carbon monoxide produced by the carbon dioxide electrolyzer and hydrogen produced by the water electrolyzer to a fuel. The fuel produced can be synthetic gasoline and/or synthetic diesel. A renewable fuel production process includes the steps of extracting carbon dioxide from atmospheric air via a carbon dioxide capture unit, converting carbon dioxide to carbon monoxide via a carbon dioxide electrolyzer, converting water to hydrogen via a water electrolyzer, and converting carbon monoxide produced via the carbon dioxide electrolyzer and H₂ produced via the water electrolyzer to a fuel. The system is also capable of simultaneously or alternatively producing a separate industrial chemical.

### CROSS-REFERENCE TO RELATED APPLICATIONS

The present application is a continuation-in-part of U.S. patent application Ser. No. 15/684,548 filed on Aug. 23, 2017, entitled “System And Process For The Production Of Renewable Fuels And Chemicals” The '548 application claimed priority benefits from U.S. provisional patent application Ser. No. 62/380,917 filed on Aug. 29, 2016, entitled “Renewal Fuel Production Systems and Process”.

The '548 application is also continuation-in-part of U.S. patent application Ser. No. 15/158,227 filed on May 18, 2016, now U.S. Pat. No. 9,945,040 issued on Apr. 17, 2018, entitled “Catalyst Layers and Electrolyzers”. The '227 application is, in turn, a continuation-in-part of U.S. patent application Ser. No. 14/704,935 filed on May 5, 2015, now U.S. Pat. No. 9,370,773 issued on Jun. 21, 2016, entitled “Ion-Conducting Membranes”.

The '548 application is also continuation-in-part of U.S. patent application Ser. No. 15/400,775 filed on Jan. 6, 2017, now U.S. Pat. No. 9,849,450 issued on Dec. 26, 2017, entitled “Ion-Conducting Membranes”. The '775 application is, in turn, a continuation-in-part of U.S. patent application Ser. No. 15/090,477, filed on Apr. 4, 2016, now U.S. Pat. No. 9,580,824 issued on Feb. 28, 2017, also entitled “Ion-Conducting Membranes”.

This application is also related to U.S. patent application Ser. No. 14/035,935, filed on Sep. 24, 2013, entitled “Devices and Processes for Carbon Dioxide Conversion into Useful Fuels and Chemicals” (now U.S. Pat. No. 9,370,733); U.S. patent application Ser. No. 12/830,338, filed on Jul. 4, 2010, entitled “Novel Catalyst Mixtures”; International application No. PCT/2011/030098 filed Mar. 25, 2011, entitled “Novel Catalyst Mixtures”; U.S. patent application Ser. No. 13/174,365, filed Jun. 30, 2011, entitled “Novel Catalyst Mixtures”; International application No. PCT/US2011/042809, filed Jul. 1, 2011, entitled “Novel Catalyst Mixtures”; U.S. patent application Ser. No. 13/530,058, filed Jun. 21, 2012, entitled “Sensors for Carbon Dioxide and Other End Uses”; International application No. PCT/US2012/043651, filed Jun. 22, 2012, entitled “Low Cost Carbon Dioxide Sensors”; and U.S. patent application Ser. No. 13/445,887, filed Apr. 12, 2012, entitled “Electrocatalysts for Carbon Dioxide Conversion”.

### STATEMENT OF GOVERNMENT INTEREST

This invention was made, at least in part, with U.S. government support under Department of Energy Contract No. DE-SC0015940. The government has certain rights in the invention.

### FIELD OF THE INVENTION

The present invention relates to a system that can easily be switched from the production of renewable fuels to the production of renewable chemicals, and that uses a CO₂ electrolyzer with a special membrane that enables CO₂ electrolysis to be accomplished at temperatures up to 120° C.

Generally, the process will involve the use of carbon dioxide and water electrolyzers to produce a mixture of carbon monoxide and hydrogen that promotes the production of fuels or that promotes the production of chemicals. The process will then use a series of reactors to produce a desired product.

### BACKGROUND OF THE INVENTION

Economic methods for producing modest quantities of renewable chemicals have been sought for years. The production of renewable chemicals would enable the U.S. to become more sustainable, but the present production costs are too high. The question is largely one of scale. The costs could be lowered if the products were made at a large scale. But the present markets are too small to justify the investment in a large-scale plant.

The situation is different for renewable fuels. The United States has made the deployment of systems and facilities to generate renewable fuels and chemicals a major priority. The Energy Independence and Security Act of 2007 (EISA) calls for the U.S. to produce 24 billion gallons (ethanol equivalent) of renewable fuel by 2017. The U.S. Environmental Protection Agency (EPA) has proposed lowering that amount to 18.8 billion gallons, because technological advances are required to produce the additional 5.2 billion gallons of renewable fuel. The situation could be even worse in 2022, when the EISA has set 36 billion gallons of renewable fuel as the production target. Stepping up the production of cellulosic ethanol cannot fill this gap because the “blend wall” (the maximum ethanol concentration allowed in fuel for gasoline-burning combustion engines) has already been reached. In this regard, ethanol is at the blend maximum of 10 parts ethanol to 90 parts gasoline to remain suitable for use in combustion engines, and there are no practical alternatives to ethanol at present. No other existing commercial scale technology can fill the gap. New technological solutions are therefore needed.

The economics associated with the production of renewable fuel are also favorable. The EISA set up a trading system for Renewable Identification Number (RIN) certificates, where one RIN is awarded for each gallon of “ethanol equivalent” fuel produced. If one produces renewable gasoline, then each gallon of gasoline would be awarded 1.56 RINs. “D3” RINs currently sell for about $2.70/gallon. California has a related low carbon fuel certificate (LCFS), by which the producer is awarded one LCFS certificate for each metric ton (MT) of CO₂ that is converted into fuel. A California LCFS certificate currently sells for $70. Calculations indicate that the sales of certificates from a 150 megawatt (MW) electrolyzer-based renewable gasoline plant would generate over $42,000,000 of revenue ($1.63/gal), thereby lowering the net cost of producing gasoline using the present system.

The net effect is the cost to produce renewable fuels approaching economic viability.

Missing at present is a way to take advantage of the growing market for renewable fuels to also produce renewable chemicals. For example, it is possible to imagine constructing a large plant that can produce either renewable fuels or renewable chemicals. In that way, the plant could serve two markets, so the cost of the plant construction could be divided over the two markets. Such a large-scale plant does not exist today, but if it could be built, it would serve the renewable fuel market and would also lower the cost of the renewable chemicals, to help that market develop.

### SUMMARY OF THE INVENTION

Shortcomings of existing systems and processes for producing quantities of renewable fuels and chemicals are overcome by a production system and process that allows convenient switching between making renewable chemicals and making renewable fuels. The system comprises:

(a) a CO₂ electrolyzer for converting CO₂ to CO;

(b) a water electrolyzer for converting H₂O to H₂;

(c) a mix point to combine the output of the CO₂ and water electrolyzers;

(d) a control unit to vary the ratio of CO₂ to H₂ according to which product is being produced; and

(e) a series of reactors for converting CO produced by the CO₂ electrolyzer and H₂ produced by the water electrolyzer to fuels, chemicals, or fuels and chemicals.

Preferably the CO₂ electrolyzer has a selectivity of at least 40%, more preferably at least 50%, still more preferably at least 60%, even more preferably at least 70%, most preferably at least 80%.

Preferably the CO₂ electrolyzer comprises an anion-conducting polymeric membrane.

In one embodiment, the anion conducting membrane comprises a terpolymer of styrene, vinylbenzyl-Rs and vinylbenzyl-Rx, where:
(a) Rs is a positively charged cyclic amine group,
(b) Rx is at least one constituent selected from the group consisting of Cl, OH and a reaction product between an OH or Cl and a species other than a simple amine or a cyclic amine, and
(c) the total weight of the vinylbenzyl-Rx groups is greater than 0.3% of the total weight of the membrane.

In an alternate preferred embodiment, the anion conducting membrane comprises a polymer blend or mixture of a copolymer consisting essentially of styrene and vinylbenzyl-Rs with at least one polymeric constituent selected from the group consisting of:
(a) a linear or substituted non-aromatic polyolefin;
(b) a polymer comprising cyclic amine groups;
(c) a polymer, excluding polystyrene, comprising at least one of a phenylene group and a phenyl group;
(d) a polyamide; and
(e) the reaction product of styrene and vinylbenzyl-Rs monomers with a crosslinking monomer having two carbon-carbon double bonds.

Rs is a preferably positively charged cyclic amine group, and the total weight of the at least one polymeric constituent in the membrane is less than the weight of the copolymer in the membrane.

Rs is preferably tetra-methyl-imidazolium.

The fuel produced by the foregoing system can be synthetic gasoline, diesel, jet fuel and/or avgas.

The chemicals produced by the foregoing system are preferably alcohols, olefins, or ethers, most preferably ethylene, propylene, or mixtures thereof.

Preferably, the CO₂ electrolyzer runs at temperatures above 25° C., preferably above 35° C., most preferably above 40° C.

Preferably, a suitable membrane for the CO₂ electrolyzer satisfies the following test:
(1) A cathode is prepared as follows:
   (a) Silver ink is made as follows. A mixture of 2 mg carbon black (for example, Vulcan XC 72RXC72, from Fuel Cell Earth), 0.2 ml of a 1% solution of the membrane polymer and 0.5 ml ethanol (Sigma-Aldrich, USA) is sonicated for 5 minutes. 100 mg of silver nanoparticles (for example, 20-40 nm, 45509, Alfa Aesar, Ward Hill, Mass.) with 1.5 ml ethanol are added and then sonicated for 5 more minutes.
   (b) The silver ink is then hand-painted onto a gas diffusion layer (for example, Sigracet 35 BC GDL, Ion Power Inc., New Castle, Del.) covering an area of 5 cm×5 cm. It is sintered at 80° C. for 15 minutes, followed by 120° C. for 15 minutes. It is then soaked in a 1 M KOH bath for 1 hour with the painted side face down.
(2) An anode is prepared as follows:
   (a) IrO₂ ink is made by mixing 100 mg of IrO₂ (Alfa Aesar) with 1 ml deionized water (18.2 Mohm Millipore), 2 ml isopropanol (3032-16, Macron) and 0.101 ml of 5% Nafion solution (1100EW, DuPont, Wilmington, Del.).
   (b) The IrO₂ ink is then hand-painted onto a 5% wet proofed carbon fiber paper (for example, TGP-H-120 5% Teflon Treated Toray Paper, from Fuel Cell Earth) covering an area of 6 cm×6 cm. Then, the carbon paper is sintered at 80° C. for 30 min.
(3) A 50-300 micrometer thick membrane of a “test” material is made by conventional means such as casting or extrusion.
(4) The membrane is sandwiched between a 3×3 cm piece of the anode material and a 2.5×2.5 cm piece of the cathode material with the metal layers on the anode and cathode facing the membrane.
(5) The membrane electrode assembly is mounted in Fuel Cell Technologies (Albuquerque, N. Mex.) 5 cm² fuel cell hardware assembly with serpentine flow fields.
(6) CO₂ humidified at 65° C. is fed into the cathode at a rate of 20 sccm and 10 mM KHCO₃ is fed into the anode flow field at a flow rate of 3 ml/min.
(7) The cell is heated to 50° C., and a power supply is connected.
(8) The cell is maintained at 3 V for 2 hours, then is switched to constant current mode at 200 mA/cm².
(9) The cell is maintained in constant current mode for at least 100 hours.
(10) Selectivity is calculated as follows:

$$Selectivity = \frac{( CO production rate )}{( CO production rate + {H}_{2} production rate )}$$

where the CO and H₂ production rates are measured in standard cubic centimeters per minute (sccm) leaving the electrolyzer. If Selectivity is greater than 90%, and the voltage to maintain 200 mA/cm² is less than 3 V, the membrane is suitable.

The series of reactors preferably includes at least 3 reactors.

The series of reactors preferably includes a first reactor that converts the CO and H₂ to methanol, then the methanol is converted dimethyl ether in a second reactor, and the dimethyl ether is converted in a third reactor to a synthetic fuel and/or a chemical.

The conversion of dimethyl ether to a synthetic fuel and/or a chemical preferably employs a zeolite catalyst such as ZSM-5 or SAPO-34.

The zeolite preferably consists of material with an SiO₂/Al₂O₃ weight ratio of 2 to 9, a Brunauer-Emmett-Teller (BET) surface of 250 to 500 m²/g, and an Na content under 200 ppm, such as the catalyst described in U.S. Pat. No. 9,174,204.

A process for the production of renewable fuel in a CO₂ collection unit for extracting CO₂ from a sustainable source:
(a) converting CO₂ to CO via a CO₂ electrolyzer;
(b) converting H₂O to H₂ via a water electrolyzer; and
(c) converting CO produced via the CO₂ electrolyzer and H₂ produced via the water electrolyzer to a fuel.

The fuel produced by the foregoing process can be synthetic gasoline and/or diesel, jet fuel and/or avgas (aviation gasoline).

The sustainable source of CO₂ can be atmospheric air or CO₂ output from a fermenter, a municipal waste treatment facility, a wood processing unit, or a landfill.

The extracted CO₂in the foregoing process is preferably substantially pure. The H₂produced in the foregoing process is also preferably substantially pure. More preferably, both the extracted CO₂ and the H₂ produced in the foregoing process are substantially pure.

### BRIEF DESCRIPTION OF THE DRAWINGS

FIG. 1 is a schematic diagram of the present renewable fuel production system.

FIG. 2 is a schematic diagram of the present renewable fuel production system configured to produce mainly gasoline.

FIG. 3 is a schematic diagram the present renewable fuel production system configured to produce mainly olefins, preferable propylene.

FIG. 4 is a schematic diagram of an alternate design of the present renewable fuel production system in which the system produces both gasoline and olefins.

### DETAILED DESCRIPTION OF ILLUSTRATIVE EMBODIMENT(S)

The present production system converts air, water, and renewable electricity into renewable fuel and/or chemicals. The system includes the following subsystems:
a CO₂ electrolyzer for converting CO₂ to CO (and O₂);
a water electrolyzer for converting H₂O to H₂ (and O₂);
a controller to adjust the ratio of CO and H₂ produced by the electrolyzers; and
a series of reactors for converting CO produced by the CO₂ electrolyzer and H₂ produced by the water electrolyzer to fuels and/or chemicals.

These subsystems have demonstrated reactant production and economic efficiencies that make their combination advantageous for the production of gasoline.

Provided immediately below is a Definitions section, where certain terms related to the process are defined specifically. Particular methods, devices, and materials are described, although any methods and materials similar or equivalent to those described herein can be used in the practice or testing of the process.

### Definitions

The term “electrochemical conversion of CO₂” as used herein refers to any electrochemical process in which carbon dioxide, carbonate, or bicarbonate is converted into another chemical substance in any step of the process.

The term “polymer electrolyte membrane” as used herein refers to both cation exchange membranes, which generally comprise polymers having multiple covalently attached negatively charged groups, and anion exchange membranes, which generally comprise polymers having multiple covalently attached positively charged groups. Typical cation exchange membranes include proton conducting membranes, such as the perfluorosulfonic acid polymer available under the trade designation NAFION from E. I. du Pont de Nemours and Company (DuPont) of Wilmington, Del.

The term “anion exchange membrane electrolyzer” as used herein refers to an electrolyzer with an anion-conducting polymer electrolyte membrane separating the anode from the cathode.

The term “liquid free cathode” refers to an electrolyzer where there are no bulk liquids in direct contact with the cathode during electrolysis. There can be a thin liquid film on or in the cathode, however, and occasional washes or rehydration of the cathode with liquids could occur.

The term “faradaic efficiency” as used herein refers to the fraction of the electrons applied to the cell that participate in reactions producing carbon-containing products.

The term “MEA” as used herein refers to a membrane electrode assembly.

The term “GC” as used herein refers to a gas chromatograph.

The term “imidazolium” as used herein refers to a positively charged ligand containing an imidazole group. This includes a bare imidazole or a substituted imidazole. Ligands of the form:

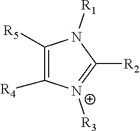

where R₁-R₅ are each independently selected from hydrogen, halides, linear alkyls, branched alkyls, cyclic alkyls, heteroalkyls, aryls, heteroaryls, alkylaryls, heteroalkylaryls, and polymers thereof, such as the vinyl benzyl copolymers described herein, are specifically included.

The term “pyridinium” as used herein refers to a positively charged ligand containing a pyridine group. This includes a bare pyridine or a substituted pyridine. Ligands of the form:

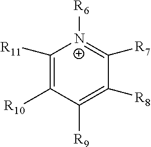

where R₆-R₁₁ are each independently selected from hydrogen, halides, linear alkyls, branched alkyls, cyclic alkyls, heteroalkyls, aryls, heteroaryls, alkylaryls, heteroalkylaryls, and polymers thereof, such as the vinyl benzyl copolymers described herein, are specifically included.

The term “phosphonium” as used herein refers to a positively charged ligand containing phosphorous. This includes substituted phosphorous. Ligands of the form:

P⁺(R₁₂R₁₃R₁₄R₁₅)

where R₁₂-R₁₅ are each independently selected from hydrogen, halides, linear alkyls, branched alkyls, cyclic alkyls, heteroalkyls, aryls, heteroaryls, alkylaryls, heteroalkylaryls, and polymers thereof, such as the vinyl benzyl copolymers described herein, are specifically included.

The term “positively charged cyclic amine” as used herein refers to a positively charged ligand containing a cyclic amine. This specifically includes imidazoliums, pyridiniums, pyrazoliums, pyrrolidiniums, pyrroliums, pyrimidiums, piperidiniums, indoliums, triaziniums, and polymers thereof, such as the vinyl benzyl copolymers described herein.

The term “PSTMIM Solution” as referred herein refers to a solution prepared as described in Specific Example 3 herein.

The term “sustainable source” as used herein refers to a source of CO₂ other than a CO₂ well or other natural CO₂ source. Sustainable sources specifically include CO₂ captured from the air, CO₂ from a fermenter, CO₂ from a municipal waste facility and CO₂ from a landfill.

The term “and/or” as used herein means “either or both”.

### SPECIFIC EXAMPLE 1

### Basic System Design

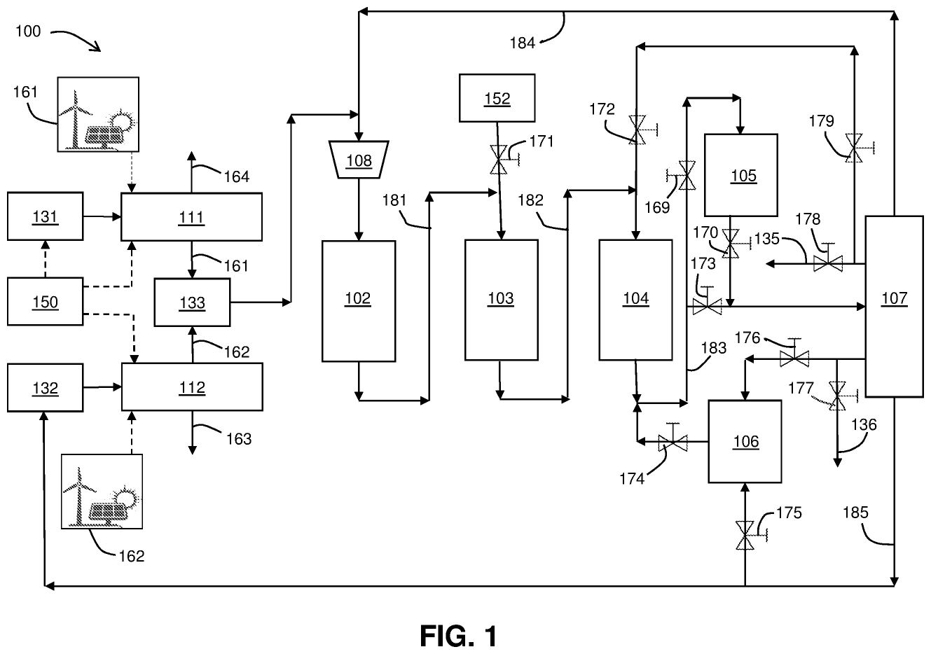

FIG. 1 is a schematic flow diagram of the present renewable fuel production system 100. System 100 includes electrolyzers 111 and 112, reactors 102, 103, 104, 105 and 106, separator 107, compressor 108, valves 169, 170, 171, 172, 173, 174, 175, 176, 177, 178 and 179, controller 150, and mix point 133. As further shown in FIG. 1, system 100 also includes a source of renewable CO₂ 131, a source of water 132, a source of bio-methanol 152, a combined CO and CO₂ stream 161 exiting electrolyzer 111 and directed to mix point 133, an H₂ stream 162 exiting electrolyzer 112 and directed to mix point 133, an O₂ outlet stream 163 exiting electrolyzer 112, and an O₂ outlet stream 164 exiting electrolyzer 111. A methanol stream 181 exits reactor 102 and is directed to the inlet stream of reactor 103. A dimethyl ether stream 182 exits reactor 103 and is directed to the inlet stream of reactor 104. A combined gasoline, propylene and tar stream 183 exits reactor 104 and is directed to the inlet stream of reactor 105 and/or to the inlet stream of separator 107. The streams exiting separator 107 include propylene exit stream 135, gasoline exit stream 136, a combined H₂, CO and CO₂ stream 184 and an H₂O stream 185. A renewable energy source 161 powers electrolyzer 111. A renewable energy source 162 powers electrolyzer 112.

Electrolyzer 111 converts CO₂ to CO via the reaction CO₂→CO+1/2O₂. A preferred design is set forth in Example 1 of co-owned U.S. Pat. No. 9,481,939.

Electrolyzer 112 converts H₂O to H₂ via the reaction H₂O→H₂+1/2O₂. A preferred design is set forth in co-owned U.S. patent application Ser. No. 15/406,909.

Controller 150 adjusts the ratio of CO, H₂, CO₂ and H₂O.

Mix point 133 is designed to mix the output streams from the CO₂ and water electrolyzers.

Reactor 102 converts mixtures of CO, CO₂ and H₂ to methanol. Reactor 102 preferably contains a Cu/ZnO catalyst such as MK-151 FENCE™ from Haldor-Topsoe (Linyi, Denmark).

Reactor 103 converts methanol to dimethyl ether. Reactor 103 preferable contains a γ-Al₂O₃ catalyst such as BASF G-250 catalyst.

Reactor 104 converts dimethyl ether to either olefins, such as propylene, or into gasoline. Reactor 104 preferably contains a zeolite catalyst such as ZSM-5 or SAPO-34. Most preferably, the zeolite consists of material with an SiO₂/Al₂O₃ weight ratio of 2 to 9, a BET surface of 250 to 500 m²/g, and an Na content under 200 ppm, such as the catalyst described in U.S. Pat. No. 9,174,204.

Reactor 105 hydrogenates durene and other tar molecules. Reactor 105 preferably contains a nickel on alumina catalyst such as Criterion KL6515, or a cobalt molybdate on alumina catalyst, such as Alfa Aesar 45579.

Reactor 106 converts the C₅⁺ molecules (molecules containing 5 or more carbons) back to CO, H₂ and light olefins via reaction with steam. Reactor 106 preferably contains either a ZSM-5 catalyst or a nickel on alumina catalyst.

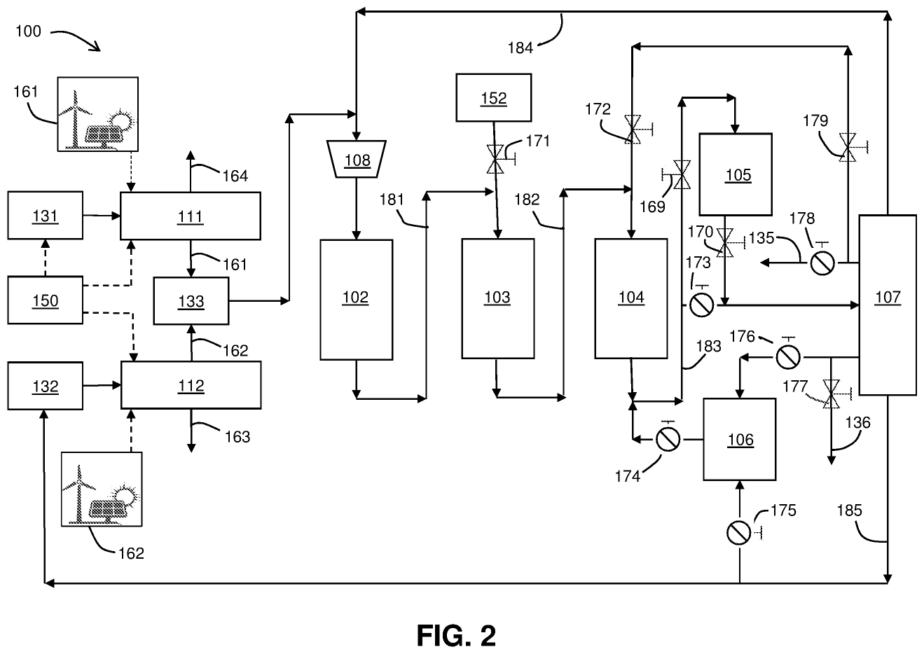

FIG. 2 illustrates operation of renewable fuel production system 100 to produce mainly fuels such as gasoline. As shown in FIG. 2, valves 173, 174, 175, 176 and 178 are closed, as depicted by the circle-and-backlash symbol ( [symbol] ) over each of those valves, and reactor 106 is shut down or placed into a regeneration cycle. In this case, the tar is hydrogenated in reactor 105 before the separation step, and olefins produced are recycled back to reactor 104 to produce more gasoline.

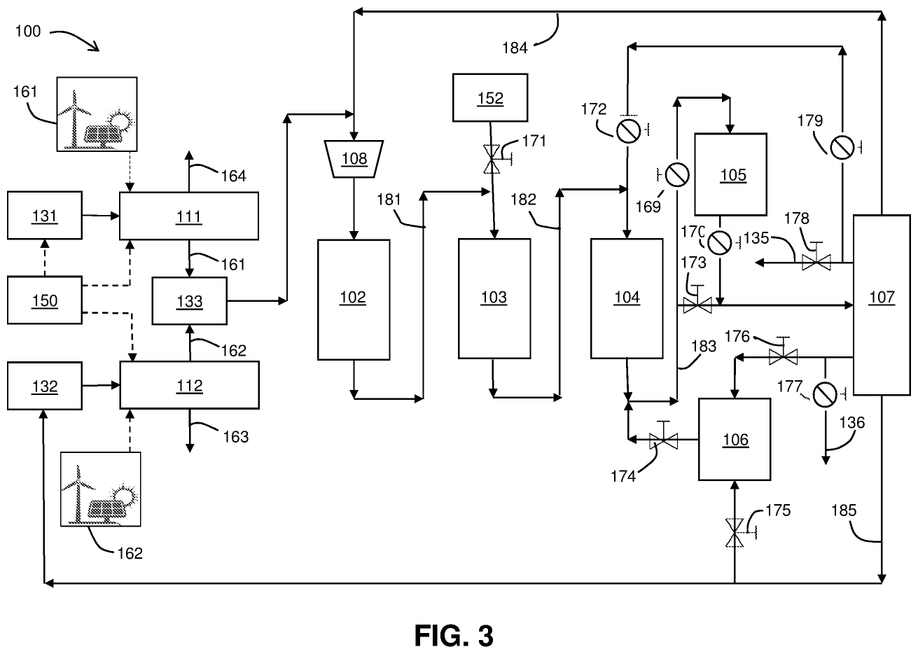

FIG. 3 shows how the device will be operated to produce mainly olefins such as propylene. In this case valves 169, 170, 172, 177 and 179 are closed, as depicted by the circle-and-backlash symbol ( [symbol] ) over each of those valves, and reactor 105 is shut down or placed into a regeneration cycle. The controller 201 adjusts the CO, CO₂ to H₂ to promote gasoline production. In this case, the tar and gasoline is sent to reactor 106 and the gasoline, tar and other hydrocarbons are cracked to produce light olefins, CO, CO₂ and H₂.

The advantages of this design are:
(a) Easy switching from making fuels to making chemicals. The chemicals have a limited market, but they are high value. Fuels have a much larger market, but they are lower value. By combining the two processes, we can take advantage of the economies of scale associated with manufacturing a high-volume product, and still also make a high value, low-volume product.
(b) Use of electrolyzers, 100 and 101, and controller 201, allows one to adjust the ratio of the CO, CO₂, H₂ and H₂O in the feed to reactor 102 to promote the production of products. For example, the preferred CO to H₂ ratio to produce gasoline is about 1:2.5, but, for example, steam methane reforming gives about 1:3.
(c) The renewable fuel production system and process described herein is carbon negative and provides energy-efficient generation of energy-dense liquid fuels or chemicals from renewable energy, water and air.

### SPECIFIC EXAMPLE 2

### Alternate System Embodiment

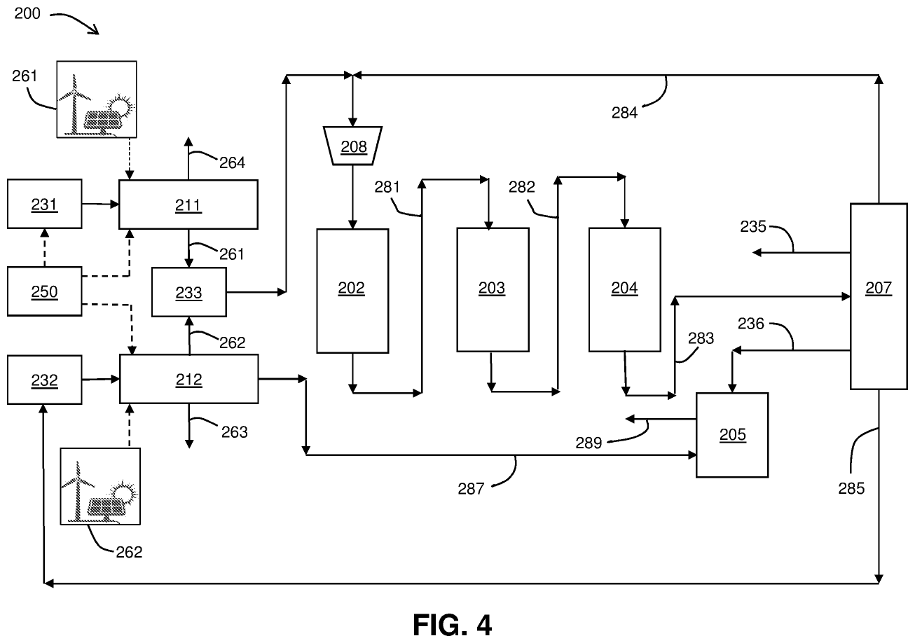

FIG. 4 shows an alternate system embodiment 200 in which both propylene and gasoline are produced. In this embodiment, the design is simplified to omit reactor 106 in system 100 shown in FIG. 1. System 200 includes electrolyzers 211 and 212, reactors 202, 203, 204 and 205, separator 207, compressor 208, controller 250, and mix point 233. As further shown in FIG. 1, system 200 also includes a source of renewable CO₂ 231, a source of water 232, a combined CO and CO₂ stream 261 exiting electrolyzer 211 and directed to mix point 233, an H₂ stream 262 exiting electrolyzer 212 and directed to mix point 233, an H₂ stream 287 exiting electrolyzer 212 and directed to reactor 205, an O₂ outlet stream 263 exiting electrolyzer 212, and an O₂ outlet stream 264 exiting electrolyzer 211. A methanol stream 281 exits reactor 202 and is directed to the inlet stream of reactor 203. A dimethyl ether stream 282 exits reactor 203 and is directed to the inlet stream of reactor 204. A combined gasoline, propylene and tar stream 283 exits reactor 204 and is directed to the inlet stream of separator 207. The streams exiting separator 207 include propylene exit stream 235, a combined gasoline and tar exit stream 236, a combined H₂, CO and CO₂ stream 284 and an H₂O stream 285. A gasoline stream 289 exits reactor 205. A renewable energy source 261 powers electrolyzer 211. A renewable energy source 262 powers electrolyzer 212.

Electrolyzer 211 converts CO₂ to CO via the reaction CO₂→CO+1/2O₂. A preferred design is set forth in Example 1 of co-owned U.S. Pat. No. 9,481,939.

Electrolyzer 212 converts H₂O to H₂ via the reaction H₂O→H₂+1/2O₂. A preferred design is set forth in co-owned U.S. patent application Ser. No. 15/406,909, published as US 2017/0233881 A1.

Controller 250 adjusts the ratio of CO, H₂, CO₂ and H₂O.

Mix point 233 is designed to mix the output streams from the CO₂ and water electrolyzers.

Reactor 202 converts mixtures of CO, CO₂ and H₂ to methanol. Reactor 202 preferably contains a Cu/ZnO catalyst such as MK-151 FENCE™ from Haldor-Topsoe (Lyngby, Denmark).

Reactor 203 converts methanol to dimethyl ether. Reactor 203 preferable contains a γ-Al₂O₃ catalyst such as BASF G-250 catalyst.

Reactor 204 converts dimethyl ether to either olefins, such as propylene, or into gasoline. Reactor 104 preferably contains a zeolite catalyst such as ZSM-5 or SAPO-34. Most preferably, the zeolite consists of material with an SiO₂/Al₂O₃ weight ratio of 2 to 9, a BET surface of 250 to 500 m²/g, and an Na content under 200 ppm, such as the catalyst described in U.S. Pat. No. 9,174,204.

Reactor 205 hydrogenates durene and other tar molecules. Reactor 205 preferably contains a nickel on alumina catalyst such as Criterion KL6515, or a cobalt molybdate on alumina catalyst, such as Alfa Aesar 45579.

### SPECIFIC EXAMPLE 3

### Improved CO₂ Electrolyzer

The objective of this example is to demonstrate that a terpolymer of styrene, vinylbenzyl-Rs and vinylbenzyl-Rx, has significant advantages as a membrane for the CO₂ electrolyzer, where
(a) Rs is a positively charged cyclic amine group,
(b) Rx is at least one constituent selected from the group consisting of Cl, OH and a reaction product between an OH or Cl and a species other than a simple amine or a cyclic amine, and
(c) the total weight of the vinylbenzyl-Rx groups is greater than 0.3% of the total weight of the membrane.

Specific Examples 1 and 2 used the carbon dioxide electrolyzer disclosed in Example 1 in the co-owned U.S. Pat. No. 9,481,939. This electrolyzer was designed to run at 25° C. One can operate the electrolyzer at higher temperatures, but the selectivity of the conversion process to CO drops with time because the membrane in Example 1 of the '939 patent degrades. As a result, the electrolyzer in Example 1 of the '939 patent cannot give stable performance at temperatures greater than 25-30° C.

There are several advantages to operating the electrolyzers between 30° C. and 120° C., preferably between 40° C. and 90° C. The reaction rate of the CO₂ conversion increases as the temperature increases. It is easier to remove heat from the electrolyzer if the electrolyzer is running at temperatures above 30° C. Pure CO has an autoignition temperature of 90° C. Mixtures might not ignite until 120° C. So, from a safety standpoint, one wishes the temperature of the electrolyzer to be below 120° C., preferably below 90° C.

It is believed that there are no current examples of a CO₂ electrolyzer operating in the temperature range of 40° C. to 120° C. The objective of this example to provide an example electrolyzer design that allows successful operation of a polymer electrolyte membrane-based CO₂ electrolyzer at higher temperatures.

First, a terpolymer membrane is prepared as described in specific Example 17 in co-owned U.S. patent application Ser. No. 15/400,775, now U.S. Pat. No. 9,849,450, as described below.

### Step 1. Production of PSTMIM Solution.

Inhibitor-free styrene was prepared by adding a volume V of styrene (Sigma-Aldrich, Saint Louis, Mo.) and a volume equal to V/4 of 4% aqueous sodium hydroxide into a separatory funnel, followed by agitating the funnel to mix the water and styrene, then decanting the styrene layer. The process was repeated five times until the water layer did not show discernible color change. The procedure was repeated using pure water instead of sodium hydroxide solution until the water layer pH was neutral. Washed styrene was put into a freezer overnight before weighing, to confirm that residual water was mainly in ice form and was then separated from styrene by filtration or decantation. 4-vinylbenzyl chloride (4-VBC) was treated in the same manner as styrene.

Poly(4-vinylbenzyl chloride-co-styrene) was then synthesized by heating a solution of inhibitor-free styrene (Sigma-Aldrich) (172.3 g, 1.65 mol) and 4-vinylbenzyl chloride (Sigma-Aldrich) (143.1 g, 0.94 mol) in chlorobenzene (Sigma-Aldrich) (250 g) at 60-65° C. in an oil bath for 22 hours under nitrogen gas with AIBN (α,α′-Azoisobutyronitrile, Sigma-Aldrich) (2.9635 g, 0.94 wt % based on the total monomers weight) as initiator. The copolymer was precipitated in methanol and washed thoroughly and dried at 60° C. overnight.

Next 1,2,4,5-tetramethylimidazole (TCI, Japan) (3.700 g, 0.0298 mol), above-synthesized poly(4-VBC-co-St) (10 g), anhydrous ethanol (17 g, Sigma-Aldrich, USA), anhydrous toluene (12.5 g, Sigma-Aldrich, USA), divinyl benzene (DVB, 0.2 g, 0.00154 mol in 1 g ethanol) and AIBN (0.00301 g in 0.97 g ethanol) were mixed under the protection of nitrogen flow. The mixture was stirred and heated to 78° C. for about 1 hour. When the solution turned clear, reaction temperature was decreased to 55° C. and maintained for 71 hours to obtain a membrane polymer.

### Step 2. Membrane Formation

The membranes were prepared by casting the polymer solutions prepared above directly onto a polyethylene terephthalate (PET) liner. The thickness of the solution on the liner was controlled by a film applicator (MTI Corporation, Richmond, Calif.) with an adjustable doctor blade. The membranes were then dried in a vacuum oven with temperature increased to 70° C. and held for 1 hour. After one more hour in the vacuum oven with temperature slowly decreased, the membrane was taken out of the oven and put into a 1 M KOH solution overnight, during which time the membrane fell from the liner. The KOH solution was changed twice, each with a few hours of immersion, to make sure the membrane chloride ions were substantially completely exchanged, so that the membranes were substantially fully converted into the hydroxide form.

A cathode material was prepared as follows. Silver ink was made as follows. A mixture of 2 mg of carbon black (Vulcan XC 72RXC72, Fuel Cell Earth), 0.2 ml of a 1% solution of the membrane polymer and 0.5 ml ethanol (Sigma-Aldrich, USA) was sonicated for 5 minutes. 100 mg of silver nanoparticles (20-40 nm, 45509, Alfa Aesar, Ward Hill, Mass.) with 1.5 ml ethanol were added and then sonicated for 5 more minutes. The silver ink was then hand-painted onto a gas diffusion layer (Sigracet 35 BC GDL, Ion Power Inc., New Castle, Del.) covering an area of 5 cm×5 cm. It was sintered at 80° C. for 15 min followed by 120° C. for 15 min. It was then soaked in a 1 M KOH bath for 1 hour with the painted side face down.

An anode material was prepared as follows. IrO₂ ink was made by mixing 100 mg of IrO₂ (Alfa Aesar) with 1 ml deionized water (18.2 Mohm Millipore), 2 ml isopropanol (3032-16, Macron) and 0.101 ml of 5% NAFION solution (1100EW, DuPont, Wilmington, Del.). The IrO₂ ink was then hand-painted onto a 5% wet proofed carbon fiber paper (TGP-H-120 5% Teflon Treated Toray Paper, Fuel Cell Earth) covering an area of 6 cm×6 cm. The ink covered carbon fiber paper was then sintered at 80° C. for 30 minutes.

The membrane was sandwiched between the a 3×3 cm piece of the anode material and a 2.5×2.5 cm piece of the cathode material with the metal layers on the anode and cathode facing the membrane, and the entire assembly was mounted in a Fuel Cell Technologies 5 cm² fuel cell hardware assembly with serpentine flow fields.

CO₂ humidified at 25° C. was fed into the cathode flow field at a rate of 20 sccm, and 10 mM KHCO₃ was fed into the anode flow field at a flow rate of 3 ml/min. Next, the cell was connected to a power supply and the cell was run at a fixed voltage of 3 V for 2 hours, then switched to constant current mode at 200 mA/cm² for 250 hours. The cell was stable for 250 hours. The selectivity was over 90%, as shown in FIG. 5 in the '775 application.

A second membrane was prepared as above and mounted in a cell as above. CO₂ humidified at 65° C. was fed into the cell at a rate of 30 sccm, and 10 mM KHCO₃ was fed into the anode flow field at a flow rate of 3 ml/min. The cell was heated to 50° C., and the power supply was connected. Again, the cell was maintained at 3 V for 2 hours, and then switched to a constant current mode at 600 mA/cm². The cell was stable for 250 hours at 600 mA/cm² with a CO selectivity over 97%.

A third membrane was prepared as above and mounted in a cell as above. CO₂ humidified at 65 ° C. was fed into the cell at a rate of 30 sccm, and 10 mM KHCO₃ was fed into the anode flow field at a flow rate of 3 ml/min. The cell was heated to 50° C., and the power supply was connected. Again, the cell was maintained at 3 V and the current was measured. Subsequently the temperature was raised to 60° C., 70° C., and 80° C. for 2 hours each, and the current was measured. Table 1 summarizes these results.

TABLE 1
Cell current density, measured as a function of temperature

| Temperature | Current mA/cm² |
|---|---|
| 25° C. | 200 |
| 50° C. | 570 |
| 60° C. | 700 |
| 70° C. | 800 |
| 80° C. | Initially 880 but dropped to 680 |

These results demonstrate that a CO₂ electrolyzer can be successfully operated at 25-80° C., preferably 50-70° C.

### SPECIFIC EXAMPLE 4

### Supported Membrane

The objective of this example is to demonstrate that a membrane comprising a polymer blend or mixture of a copolymer consisting essentially of styrene and vinylbenzyl-Rₛ with at least one polymeric constituent selected from the group consisting of:
(a) a linear or substituted non-aromatic polyolefin;
(b) a polymer comprising cyclic amine groups;
(c) a polymer, excluding polystyrene, comprising at least one of a phenylene group and a phenyl group;
(d) a polyamide; and
(e) the reaction product of styrene and vinylbenzyl-Rₛ monomers with a crosslinking monomer having two carbon-carbon double bonds,
   wherein Rₛ is a positively charged cyclic amine group, and wherein the total weight of the at least one polymeric constituent in the membrane is less than the weight of the copolymer in the membrane, as described in co-owned U.S. Pat. No. 9,580,824.

Step 1. A PSTMIM solution was prepared as described in Specific Example 3.

Step 2. The PSTMIM solution was diluted to 20% solids with ethanol.

Step 3. A BKY (Geretsried, Germany) Automatic Film Applicator L was used to cast a thin film of the polymer solution onto a polypropylene backing sheet (Home Depot, Atlanta, Ga.) using a doctor blade. The solution was allowed to dry in ambient environment for 30 minutes to yield an approximately 15 micrometer thick polymer film.

Step 4. Next, a 10 μm thick porous expanded polytetrafluoroethylene (ePTFE) film (Philips Scientific Inc., Rock Hill, S.C.) was submerged for 30 minutes in a bath of ethanol to activate its surface for better wettability. The porous ePTFE film was then laid carefully taut over the deposited polymer film. The ePTFE film was also stretched in both x and y directions to fully open its pore structure as it was laid over the polymer film.)

Step 5. A 15 μm layer of the PSTMIM polymer solution was deposited on top of the ePTFE. The polymer film was left to settle for 15 minutes in ambient conditions before the whole reinforced membrane was placed in an oven at 65° C. for 60 minutes to improve adhesion of the polymer with the ePTFE. After the heating step, the membrane was then separated from the polypropylene backing sheet with the help of a razor blade and tweezers, and then activated in 1 M KOH, as described in Specific Example 3.

The resultant membrane was mounted in a cell and tested as in Specific Example 3. Table 2 shows the results of these experiments.

TABLE 2
The current density measured as a function of temperature

| Temperature | Current mA/cm² |
|---|---|
| 50° C. | 400 |
| 60° C. | 440 |
| 70° C. | 540 |
| 80° C. | 700 |
| 90° C. | 800 |

These results demonstrate that a CO₂ electrolyzer can be successfully operated at 25-90° C. Temperatures up to 120° C. are also viable if the electrolyzer is pressurized.

The specific order or hierarchy of steps in the methods and/or processes disclosed herein are examples of exemplary approaches. Based upon design preferences, the specific order or hierarchy of steps in the method can be rearranged while remaining within the disclosed subject matter. The accompanying method claims present elements of the various steps in a sample order, and are not necessarily meant to be limited to the specific order or hierarchy presented.

Numerical value ranges recited herein include all values from the lower value to the upper value in increments of one unit, provided that there is a separation of at least two units between a lower value and a higher value. As an example, if it is stated that the concentration of a component or value of a process variable such as, for example, size, angle, pressure, time and the like, is, for example, from 1 to 98, specifically from 20 to 80, more specifically from 30 to 70, it is intended that values such as 15 to 85, 22 to 68, 43 to 51, 30 to 32, and the like, are expressly enumerated in this specification. For values that are less than one, one unit is considered to be 0.0001, 0.001, 0.01 or 0.1 as appropriate. These are only examples of what is specifically intended and all possible combinations of numerical values between the lowest value and the highest value are to be treated in a similar manner.

While particular elements, embodiments and applications of the present invention have been shown and described, it will be understood, that the invention is not limited thereto, since modifications can be made by those skilled in the art without departing from the scope of the present disclosure, particularly in light of the foregoing teachings.

What is claimed is:

1. A system for the manufacture of renewable fuels and/or renewable chemicals comprising:
(a) a CO₂ electrolyzer for converting CO₂ to CO and O₂, wherein said CO₂ electrolyzer has a CO selectivity of at least 40%;
(b) a separate water electrolyzer for converting H₂O to H₂ and O₂;
(c) a control unit for directing signals to each of said electrolyzers to vary the ratio of CO to H₂ produced by said electrolyzers; and
(d) a series of reactors to convert the CO/H₂ mixture to fuels and/or chemicals,
wherein said CO₂ electrolyzer operates in the temperature range of 40° C. to 120° C.

2. The system of claim 1 wherein the selectivity is at least 50%.

3. The system of claim 2 wherein the selectivity is at least 60%.

4. The system of claim 3 wherein the selectivity is at least 70%.

5. The system of claim 4 wherein the selectivity is at least 80%.

6. The system in claim 1, wherein the system is capable of producing at least one fuel and at least one chemical.

7. The system of claim 6, wherein said fuel is at least one of synthetic gasoline, synthetic diesel, synthetic avgas, and a blend-stock therefor.

8. The system of claim 6, wherein said chemical is at least one of methanol, dimethylether, ethanol and propylene.

9. The system of claim 1, wherein said series of reactors comprises at least 3 reactors.

10. The system of claim 9, wherein a first reactor of said series of reactors converts the CO and H₂ to methanol, wherein a second reactor coverts methanol to dimethyl ether, and wherein a third reactor converts dimethyl ether to at least one of a synthetic fuel and a chemical.

11. The system of claim 10, wherein the reactor that converts dimethyl ether to a synthetic fuel or chemical comprises a zeolite catalyst.

12. The system of claim 11, wherein said zeolite catalyst is a micropore zeolite catalyst.

13. The system of claim 12, wherein the zeolite catalyst has an SiO₂/Al₂O₃ weight ratio of 2 to 9, a BET surface of 250 to 500 m²/g, and an Na content under 200 ppm.

14. The system of claim 1, wherein said CO₂ electrolyzer comprises:
(a) a cathode prepared as follows: Silver ink is made by mixing 2 mg carbon black, 0.2 ml of a 1% solution of the membrane polymer and 0.5 ml ethanol is sonicated for 5 minutes; 100 mg of silver nanoparticles (20-40 nm) with 1.5 ml ethanol is added and then sonicated for 5 more minutes; the silver ink is then hand-painted onto a gas diffusion layer covering an area of 5 cm×5 cm; the gas diffusion layer is sintered at 80° C. for 15 min followed by 120° C. for 15 minutes; the gas diffusion layer is then soaked in a 1 M KOH bath for 1 hour with the painted side face down; and
(b) an anode prepared as follows: IrO₂ ink is made by mixing 100 mg of IrO₂ with 1 ml deionized water, 2 ml isopropanol and 0.101 ml of 5% NAFION solution; the IrO₂ ink is then hand-painted onto a 5% wet proofed carbon fiber paper covering an area of 6 cm×6 cm; then, it is sintered at 80° C. for 30 minutes;
(c) an anion-conducting polymeric membrane interposed between the inked sides of said cathode and said anode to form a membrane electrode assembly, said membrane electrode assembly mounted in a fuel cell hardware assembly with serpentine reactant flow field channels, said membrane meeting the following test:
when (i) CO₂ humidified at 65° C. is fed into the cathode at a rate of 20 sccm and 10 mM KHCO₃ is fed into the anode flow field at a flow rate of 3 ml/min, (ii) the cell is heated to 50° C., (iii) a power supply interconnects said cathode and said anode, (iv) the cell is maintained at 3 V for 2 hours, and then switched to constant current mode at 200 mA/cm²; (v) the cell is maintained in constant current mode for at least 100 hours, (vi) selectivity is calculated as follows:
$$Selectivity = \frac{( CO production rate )}{( CO production rate + {H}_{2} production rate )}$$
and (vii) CO and H₂ production rates are measured in standard cubic centimeters per minute exiting the cell, selectivity is greater than 40%, and the voltage to maintain 200 mA/cm² is less than 3 V.
